(19) United States
(12) Patent Application Publication
(10) Pub. No.: US 2009/0092987 A1
(43) Pub. Date: Apr. 9, 2009

(54) Polymorphic Nucleic Acids Associated With Colorectal Cancer And Uses Thereof

(75) Inventors: Stephen B. Gruber, Ann Arbor, MI (US); Gad Rennert, Haifa (IL); Victor Moreno, Sant Joan Despi (ES); Laura Rozek, Ann Arbor, MI (US)

Correspondence Address: Casimir Jones, S.C., 440 Science Drive, Suite 203, Madison, WI, 53711, US

(73) Assignee: THE REGENTS OF THE UNIVERSITY OF MICHIGAN, Ann Arbor, MI (US)

(21) Appl. No.: 12/246,246

(22) Filed: Oct. 6, 2008

Related U.S. Application Data
(60) Provisional application No. 60/977,888, filed on Oct. 5, 2007.

(51) Int. Cl. C12Q 1/68

(52) U.S. Cl. 435/6

(57) ABSTRACT

The present invention provides compositions and methods for research, diagnostic, drug screening, and therapeutic applications related to colorectal cancer and related conditions. In particular, the present invention provides genetic variations in or associated with one or more of the STK38L, GPR45, TGFBRAP1, PADI3, IBRDC1, and/or GRK5 genes as being associated with such conditions.

### CROSS-REFERENCE TO RELATED APPLICATIONS

This application claims priority to U.S. Provisional Application Ser. No. 60/977,888, filed Oct. 5, 2007, which is herein incorporated by reference in its entirety.

### STATEMENT REGARDING FEDERALLY SPONSORED RESEARCH OR DEVELOPMENT

This invention was made with government support under Grant No. CA081488 awarded by the National Cancer Institute. The government has certain rights in the invention.

### FIELD OF THE INVENTION

The present invention provides compositions and methods for research, diagnostic, drug screening, and therapeutic applications related to colorectal cancer and related conditions. In particular, the present invention provides genetic variations in or associated with one or more of the STK38L, GPR45, TGFBRAP1, PADI3, IBRDC1, and GRK5 genes as being associated with such conditions.

### BACKGROUND OF THE INVENTION

Colorectal cancer is the third most common cancer in both men and women in the United States. Risk factors include age, a diet rich in fat and cholesterol, inflammatory bowel disease (especially ulcerative colitis), and genetic predisposition, including hereditary polyposis and nonpolyposis syndromes.

If detected early, colorectal cancer is curable by surgery. Adjuvant chemotherapy can prolong survival in disease that has reached the lymph nodes. Both systemic and locoregional therapy have a role in patients with metastatic colon cancer. Radiotherapy is used in cases of rectal cancer to reduce the risk of local recurrence.

Long-term survival correlates with stage of disease in colorectal cancer. Progress has been made in understanding the molecular basis of colorectal cancer predisposition and progression. Efforts are underway to develop better screening strategies, chemopreventive approaches, and novel therapies to improve patient survival rates and to minimize toxicity. Despite all efforts, colorectal cancer remains the third leading cause of death from cancer in the United States.

What is needed is a better understanding of the pathophysiology, genetics and biochemistry underlying colorectal cancer and associated conditions. Additionally, improved methods for detecting colorectal cancer, and detecting risk of developing colorectal cancer, are needed.

### SUMMARY OF THE INVENTION

The present invention provides compositions and methods for research, diagnostic, drug screening, and therapeutic applications related to colorectal cancer and related conditions. In particular, the present invention provides genetic variations in or associated with one or more of the STK38L, GPR45, TGFBRAP1, PADI3, IBRDC1, and GRK5 genes as being associated with such conditions.

Experiments conducted during the course of development of the embodiments for the present invention demonstrated genetic variations associated with one or more of the STK38L, GPR45, TGFBRAP1,PADI3, IBRDC1, and GRK5 genes as being associated with colorectal cancer. In particular, genetic markers associated with the genes demonstrated a predictive correlation to disease status. For example, it was shown that individuals having one or more single nucleotide polymorphisms selected from rsID 544670, rsID 17029, rsID 10194088, rsID 2977269, rsID 883992 and rsID 7896882 have an increased susceptibility to colorectal cancer, and that individuals having one or more SNPs selected from rsID 16931815, and rsID 10210149 have a decreased susceptibility to colorectal cancer.

Accordingly, in some embodiments, the present invention provides methods and compositions, systems and kits for the determination of genetic variation within or associated with a subject's STK38L, GPR45, TGFBRAP1, PADI3, IBRDC1, and/or GRK5 genes. The present invention is not limited to a particular type of genetic variation. Examples of genetic variation include, but are not limited to, single nucleotide polymorphisms (SNPs), mutations, insertions, deletions, altered (e.g., increased, decreased) gene copy number, microsatellite instability, altered (e.g., increased, decreased) gene expression, and heterozygosity. In some embodiments, the results are used to determine an individual's susceptibility to developing cancer (e.g., colorectal cancer) or related diseases or conditions. In some embodiments, the results are used to assess or predict progression of a disease.

For example, in some embodiments, the present invention provides a method of characterizing a disease (e.g., colorectal cancer) in a subject, comprising providing a sample from a subject; and determining genetic variation (e.g., SNPs, mutations, insertions, deletions, altered (e.g., increased, decreased) gene copy number, microsatellite instability, altered (e.g., increased, decreased) gene expression, and heterozygosity) within or associated with one or more of the subject's STK38L, GPR45, TGFBRAP1, PADI3, IBRDC1, and GRK5 genes to determine the subject's susceptibility to cancer. In some embodiments, the determining genetic variation within or associated with one or more of the subject's STK38L, GPR45, TGFBRAP1, PADI3, IBRDC1, and GRK5 genes comprises the use of a nucleic acid based detection. In some embodiments, the subject is not previously diagnosed with cancer (e.g., colorectal cancer). In some embodiments, the characterizing comprises determining a risk for developing such a disease (e.g., colorectal cancer).

The present invention further provides a method, comprising providing a sample from a subject; detecting the presence of one or more single nucleotide polymorphisms selected from the group consisting of, for example, rsID 544670, rsID 16931815, rsID 10210149, rsID 17029, rsID 10194088, rsID 2977269, rsID 883992, and rsID 7896882 or other genetic markers in linkage disequilibrium with any of the above referenced SNPs; and determining the subject's risk of developing cancer (e.g., increased risk, decreased risk) based on the presence or absence of such single nucleotide polymorphisms. In some embodiments, determining the presence of such single nucleotide polymorphisms comprises the use of a nucleic acid based detection assay.

In some embodiments, the present invention provides a kit for determining a subject's risk of developing cancer (e.g., colorectal cancer), comprising a detection assay, wherein the detection assay is configured to specifically detect genetic variation (e.g., SNPs, mutations, insertions, deletions, altered (e.g., increased, decreased) gene copy number, microsatellite instability, altered (e.g., increased, decreased) gene expression, and heterozygosity) within or associated with one or more of the subject's STK38L, GPR45, TGFBRAP1, PADI3, IBRDC1, and/or GRK5 genes. In some embodiments, the kit comprises one or more reagents sufficient, necessary or useful for carrying out a detection assay.

In some embodiments, the present invention provides a method of screening compounds, comprising: providing a cell comprising one or more STK38L, GPR45, TGFBRAP1, PADI3, IBRDC1, and GRK5 genes; and one or more test compounds; and contacting the cell with the test compound; and detecting the presence of an altered level of expression of the STK38L, GPR45, TGFBRAP1, PADI3, IBRDC1, and/or GRK5 gene in the presence of the test compound relative to the level in the absence of the test compound. In some embodiments, the cell is in an animal. In some embodiments, the animal is a non-human mammal. In some embodiments, the non-human mammal is a transgenic non-human mammal. In other embodiments, the animal is a human.

### DEFINITIONS

To facilitate an understanding of the present invention, a number of terms and phrases are defined below:

As used herein, the term “single nucleotide polymorphism” or “SNP”, refers to any position along a nucleotide sequence that has one or more variant nucleotides. Single nucleotide polymorphisms (SNPs) are the most common form of DNA sequence variation found in the human genome and are generally defined as a difference from the baseline reference DNA sequence which has been produced as part of the Human Genome Project or as a difference found between a subset of individuals drawn from the population at large. SNPs occur at an average rate of approximately 1 SNP/1000 base pairs when comparing any two randomly chosen human chromosomes. Extremely rare SNPs can be identified which may be restricted to a specific individual or family, or conversely can be found to be extremely common in the general population (present in many unrelated individuals). SNPs can arise due to errors in DNA replication (i.e., spontaneously) or due to mutagenic agents (i.e., from a specific DNA damaging material) and can be transmitted during reproduction of the organism to subsequent generations of individuals.

As used herein, the term “linkage disequilibrium” refers to single nucleotide polymorphisms or other genetic markers where the genotypes are correlated between these markers. Several statistical measures can be used to quantify this relationship (i.e. D′, r², etc) reference (See e.g., Devlin and Risch 1995 Sep. 20; 29(2):311-22; herein incorporated by reference in its entirety). In some embodiments, the marker pair is considered to be in linkage disequilibrium if r²>0.5,

As used herein, the term “haplotype” refers to a group of closely linked alleles that are inherited together.

As used herein, the term “allele” refers to a variant form of a given sequence (e.g., including but not limited to, genes containing one or more SNPs). A large number of genes are present in multiple allelic forms in a population. A diploid organism carrying two different alleles of a gene is said to be heterozygous for that gene, whereas a homozygote carries two copies of the same allele.

As used herein, the term “linkage” refers to the proximity of two or more markers (e.g., genes, SNPs) on a chromosome.

As used herein, the term “allele frequency” refers to the frequency of occurrence of a given allele in given population (e.g., a specific gender, race, or ethnic group). Certain populations may contain a given allele within a higher percent of its members than other populations. For example, a particular mutation in the breast cancer gene called BRCA1 was found to be present in one percent of the general Jewish population. In comparison, the percentage of people in the general U.S. population that have any mutation in BRCA1 has been estimated to be between 0.1 to 0.6 percent. Two additional mutations, one in the BRCA1 gene and one in another breast cancer gene called BRCA2, have a greater prevalence in the Ashkenazi Jewish population, bringing the overall risk for carrying one of these three mutations to 2.3 percent.

As used herein, the term “in silico analysis” refers to analysis performed using computer processors and computer memory. For example, “in silico SNP analysis” refers to the analysis of SNP data using computer processors and memory.

As used herein, the term “genotype” refers to the actual genetic make-up of an organism (e.g., in terms of the particular alleles carried at a genetic locus). Expression of the genotype gives rise to an organism's physical appearance and characteristics—the “phenotype.”

As used herein, the term “locus” refers to the position of a gene or any other characterized sequence on a chromosome.

As used herein the term “disease” or “disease state” refers to a deviation from the condition regarded as normal or average for members of a species, and which is detrimental to an affected individual under conditions that are not inimical to the majority of individuals of that species (e.g., the presence of colorectal cancer and related symptoms).

As used herein, the term “treatment” in reference to a medical course of action refer to steps or actions taken with respect to an affected individual as a consequence of a suspected, anticipated, or existing disease state, or wherein there is a risk or suspected risk of a disease state. Treatment may be provided in anticipation of or in response to a disease state or suspicion of a disease state, and may include, but is not limited to preventative, ameliorative, palliative or curative steps. The term “therapy” refers to a particular course of treatment.

The term “gene” (e.g., STK38L, GPR45, TGFBRAP1, PADI3, IBRDC1, and/or GRK5 gene) refers to a nucleic acid (e.g., DNA) sequence that comprises coding sequences necessary for the production of a polypeptide, RNA (e.g., rRNA, tRNA, etc.), or precursor. The polypeptide, RNA, or precursor can be encoded by a full length coding sequence or by any portion of the coding sequence so long as the desired activity or functional properties (e.g., ligand binding, signal transduction, etc.) of the full-length or fragment are retained. The term also encompasses the coding region of a structural gene, including sequences located adjacent to the coding region on both the 5′ and 3′ ends. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments included when a gene is transcribed into heterogeneous nuclear RNA (hnRNA); introns may contain regulatory elements such as enhancers. Introns are removed or “spliced out” from the nuclear or primary transcript; introns therefore are generally absent in the messenger RNA (mRNA) transcript. The mRNA functions during translation to specify the sequence or order of amino acids in a nascent polypeptide. Variations (e.g., mutations, SNPS, insertions, deletions) in transcribed portions of genes are reflected in, and can generally be detected in corresponding portions of the produced RNAs (e.g., hnRNAs, mRNAs, rRNAs, tRNAs).

Where the phrase “amino acid sequence” is recited herein to refer to an amino acid sequence of a naturally occurring protein molecule, amino acid sequence and like terms, such as polypeptide or protein are not meant to limit the amino acid sequence to the complete, native amino acid sequence associated with the recited protein molecule.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified,” “mutant,” and “variant” refer to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. In this case, the DNA sequence thus codes for the amino acid sequence.

DNA and RNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotides or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the terms “an oligonucleotide having a nucleotide sequence encoding a gene” and “polynucleotide having a nucleotide sequence encoding a gene,” means a nucleic acid sequence comprising the coding region of a gene or, in other words, the nucleic acid sequence that encodes a gene product. The coding region may be present in either a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

The term “homology” refers to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous.” The term “inhibition of binding,” when used in reference to nucleic acid binding, refers to inhibition of binding caused by competition of homologous sequences for binding to a target sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i. e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41 (% G+C), when a nucleic acid is in aqueous solution at 1 M NaCl (See e.g., Anderson and Young, Quantitative Filter Hybridization, in Nucleic Acid Hybridization [1985]; herein incorporated by reference in its entirety). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄ H₂0 and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42 C when a probe of about 500 nucleotides in length is employed.

“Medium stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42 C when a probe of about 500 nucleotides in length is employed.

“Low stringency conditions” comprise conditions equivalent to binding or hybridization at 42 C in a solution consisting of 5×SSPE (43.8 g/l NaCl, 6.9 g/l NaH₂PO₄ H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 g/ml denatured salmon sperm DNA followed by washing in a solution comprising 5× SSPE, 0.1% SDS at 42 C when a probe of about 500 nucleotides in length is employed.

The following terms are used to describe the sequence relationships between two or more polynucleotides: “reference sequence,” “sequence identity,” “percentage of sequence identity,” and “substantial identity.” A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window,” as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i. e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman [Smith and Waterman, Adv. Appl. Math. 2: 482 (1981); herein incorporated by reference in its entirety] by the homology alignment algorithm of Needleman and Wunsch [Needleman and Wunsch, J. Mol. Biol. 48:443 (1970); herein incorporated by reference in its entirety], by the search for similarity method of Pearson and Lipman [Pearson and Lipman, Proc. Natl. Acad. Sci. (U.S.A.) 85:2444 (1988); herein incorporated by reference in its entirety], by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i. e., the window size), and multiplying the result by 100 to yield the percentage of sequence identity.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acids encoding a polypeptide include, by way of example, such nucleic acid in cells ordinarily expressing the polypeptide where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (e.g., 10 nucleotides, 11, . . . , 20, . . . ).

As used herein, the term “purified” or “to purify” refers to the removal of contaminants from a sample. As used herein, the term “purified” refers to molecules (e.g., nucleic or amino acid sequences) that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

The term “test compound” refers to any chemical entity, pharmaceutical, drug, and the like that are tested in an assay (e.g., a drug screening assay) for any desired activity (e.g., including but not limited to, the ability to treat or prevent a disease, illness, sickness, or disorder of bodily function, or otherwise alter the physiological or cellular status of a sample). Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “sample” as used herein is used in its broadest sense. A sample suspected of containing a human chromosome or sequences associated with a human chromosome may comprise a cell, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

The term “label” as used herein refers to any atom or molecule that can be used to provide a detectable (preferably quantifiable) effect, and that can be attached to a nucleic acid or protein. Labels include but are not limited to dyes; radiolabels such as ³²P; binding moieties such as biotin; haptens such as digoxgenin; luminogenic, phosphorescent or fluorogenic moieties; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, and the like. A label may be a charged moiety (positive or negative charge) or alternatively, may be charge neutral. Labels can include or consist of nucleic acid or protein sequence, so long as the sequence comprising the label is detectable.

The term “signal” as used herein refers to any detectable effect, such as would be caused or provided by a label or an assay reaction.

As used herein, the term “detector” refers to a system or component of a system, e.g., an instrument (e.g. a camera, fluorimeter, charge-coupled device, scintillation counter, etc) or a reactive medium (X-ray or camera film, pH indicator, etc.), that can convey to a user or to another component of a system (e.g., a computer or controller) the presence of a signal or effect. A detector can be a photometric or spectrophotometric system, which can detect ultraviolet, visible or infrared light, including fluorescence or chemiluminescence; a radiation detection system; a spectroscopic system such as nuclear magnetic resonance spectroscopy, mass spectrometry or surface enhanced Raman spectrometry; a system such as gel or capillary electrophoresis or gel exclusion chromatography; or other detection system known in the art, or combinations thereof.

The term “detection” as used herein refers to quantitatively or qualitatively identifying an analyte (e.g., DNA, RNA or a protein) within a sample. The term “detection assay” as used herein refers to a kit, test, or procedure performed for the purpose of detecting an analyte nucleic acid within a sample. Detection assays produce a detectable signal or effect when performed in the presence of the target analyte, and include but are not limited to assays incorporating the processes of hybridization, nucleic acid cleavage (e.g., exo- or endonuclease), nucleic acid amplification, nucleotide sequencing, primer extension, or nucleic acid ligation.

The terms “assay data” and “test result data” as used herein refer to data collected from performance of an assay (e.g., to detect or quantitate a gene, SNP or an RNA). Test result data may be in any form, i.e., it may be raw assay data or analyzed assay data (e.g., previously analyzed by a different process). Collected data that has not been further processed or analyzed is referred to herein as “raw” assay data (e.g., a number corresponding to a measurement of signal, such as a fluorescence signal from a spot on a chip or a reaction vessel, or a number corresponding to measurement of a peak, such as peak height or area, as from, for example, a mass spectrometer, HPLC or capillary separation device), while assay data that has been processed through a further step or analysis (e.g., normalized, compared, or otherwise processed by a calculation) is referred to as “analyzed assay data” or “output assay data”.

As used herein, the term “database” refers to collections of information (e.g., data) arranged for ease of retrieval, for example, stored in a computer memory. A “genomic information database” is a database comprising genomic information, including, but not limited to, polymorphism information (i.e., information pertaining to genetic polymorphisms), genome information (i.e., genomic information), linkage information (i.e., information pertaining to the physical location of a nucleic acid sequence with respect to another nucleic acid sequence, e.g., in a chromosome), and disease association information (i.e., information correlating the presence of or susceptibility to a disease to a physical trait of a subject, e.g., an allele of a subject). “Database information” refers to information to be sent to a databases, stored in a database, processed in a database, or retrieved from a database. “Sequence database information” refers to database information pertaining to nucleic acid sequences. As used herein, the term “distinct sequence databases” refers to two or more databases that contain different information than one another. For example, the dbSNP and GenBank databases are distinct sequence databases because each contains information not found in the other.

As used herein, the term “detection assay component” refers to a component of a system capable of performing a detection assay. Detection assay components include, but are not limited to, hybridization probes, buffers, and the like.

As used herein, the term “a detection assays configured for target detection” refers to a collection of assay components that are capable of producing a detectable signal when carried out using the target nucleic acid. For example, a detection assay that has empirically been demonstrated to detect a particular single nucleotide polymorphism is considered a detection assay configured for target detection.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to a delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

### DETAILED DESCRIPTION OF THE INVENTION

The vast majority of colorectal cancers are adenocarcinomas, which arise from preexisting adenomatous polyps that develop in the normal colonic mucosa. This adenoma-carcinoma sequence is a well-characterized clinical and histopathologic series of events with which discrete molecular genetic alterations have been associated. A number of critically important genetic alterations that contribute, through their multiplicity over many years, to the eventual development of colorectal cancer have been identified. An early event appears to involve the APC (adenomatous polyposis coli) gene, which is mutated in individuals affected by familial adenomatous polyposis (FAP). The protein encoded by the APC gene targets the degradation of beta-catenin, a protein component of a transcriptional complex that activates growth-promoting oncogenes, such as cyclin D1 or c-myc. APC mutations are very common in sporadic colorectal cancer, and beta-catenin mutations have also been identified.

DNA methylation changes are a relatively early event and have been detected at the polyp stage. Colorectal cancers and polyps have an imbalance in genomic DNA methylation, with global hypomethylation and regional hypermethylation. Hypomethylation can lead to oncogene activation, whereas hypermethylation can lead to silencing of tumor suppressor genes. ras gene mutations are observed commonly in larger polyps but not smaller polyps, suggesting a role for this oncogene in polyp growth.

Chromosome arm 18q deletions are a later event associated with cancer development. These deletions likely involve the targets DPC4 (a gene deleted in pancreatic cancer and involved in the transforming growth factor [TGF]-beta growth-inhibitory signaling pathway) and DCC (a gene frequently deleted in colon cancer). Chromosome arm 17p losses and tumor suppressor p53 mutations are common late events in colon cancer. Bc12 overexpression leading to inhibition of cell death signaling has been observed as a relatively early event in colorectal cancer development. 18q deletions detected in Dukes stage B colon cancers have been associated with an increased risk of recurrence following surgery.

Experiments conducted during the course of development of embodiments for the present invention performed a whole genome association study of colorectal cancer and identified markers for susceptibility to colorectal cancer. The candidates were validated in a separate set of colorectal cancer cases and controls. Genetic variation in 8 markers, including 6 genes, were shown to modify the risk of colorectal cancer. It was shown that genetic variation at each of the markers and associated genes predicted risk of colorectal cancer and could be used for diagnosis and prognosis. Genetic variation in and around genes STK38L, GPR45, TGFBRAP1, PADI3, IBRDC1, and/or GRK5 was shown to be significantly predictive of risk of colorectal cancer.

Accordingly, in some embodiments, the present invention provides methods and compositions for the determination of genetic variation within or associated with one or more of a subject's STK38L, GPR45, TGFBRAP1, PADI3, IBRDC1, and/or GRK5 genes. Examples of genetic variation include, but are not limited to, single nucleotide polymorphisms (SNPs), mutations, insertions, deletions, altered (e.g., increased, decreased) gene copy number, microsatellite instability, altered (e.g., increased, decreased) gene expression, and heterozygosity. In some embodiments, the results are used to determine an individual's susceptibility to cancer (e.g., colorectal cancer) or to particular cancer therapies, treatment, or interventions. The present invention further provides drug-screening methods to screen for compounds that alter the expression or activity of STK38L, GPR45, TGFBRAP1, PADI3, IBRDC1, and/or GRK5 polypeptides (e.g., polymorphic STK38L, GPR45, TGFBRAP1, PADI3, IBRDC1, and/or GRK5 polypeptides).

In some embodiments, one or more markers of the present invention is analyzed during a routine physical examination. For example, a sample may be collected during a routing preventative colorectal cancer screening (e.g., alone or in the place of a colonoscopy). In some embodiments, screening is carried out on subjects in a risk category (e.g., based on age, family history, prior disease, etc.). In some embodiments, the markers of the invention are used as an initial screening. Suggestive results may be followed up with additional diagnostic testing, monitoring, and/or therapy.

### I. Detection Assays

The present invention provides comprehensive systems and methods for the identification of genetic variation within or associated with the STK38L, GPR45, TGFBRAP1, PADI3, IBRDC1, and/or GRK5 genes. Exemplary detection assays are described below. It is contemplated that the below described detection assays can be configured for multiplex detection (e.g., detecting two or more of the markers or one or more of the markers with one or more other known markers).

There are a wide variety of detection technologies available for determining the sequence, structure, and/or expression of a target nucleic acid at one or more locations. Many of these techniques require the use of an oligonucleotide to hybridize to the target. Depending on the assay used, the oligonucleotide is then cleaved, elongated, ligated, disassociated, or otherwise altered, wherein its behavior in the assay is monitored as a means for characterizing the sequence of the target nucleic acid. Examples of detection assays include, but are not limited to, nucleic acid sequencing technologies, enzymatic assays (e.g., Taqman), NASBA, PCR, TMA, hybridization assays, bead-bead assays, microarrays, mass spectroscopy-based assays, and the like.

### II. Data Analysis

In some embodiments, a computer-based analysis program is used to translate the raw data generated by the detection assay (e.g., the genotype of a STK38L, GPR45, TGFBRAP1, PADI3, IBRDC1, and/or GRK5 gene) into data of predictive value for a clinician. The clinician can access the predictive data using any suitable means. Thus, in some embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (e.g., STK38L, GPR45, TGFBRAP1, PADI3, IBRDC1, and/or GRK5 haplotype), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., likelihood of developing colorectal cancer or related complications) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

### EXPERIMENTAL

The following examples are provided in order to demonstrate and further illustrate certain embodiments and aspects of the present invention and are not to be construed as limiting the scope thereof.

### EXAMPLE I

This Example describes how genetic variations in one or more of the STK38L, GPR45, TGFBRAP1, PADI3, IBRDC1, and/or GRK5 genes were determined to be associated with a risk for developing colorectal cancer. A whole genome association study was conducted to identify candidate colorectal cancer markers for susceptibility to colorectal cancer. The whole genome association study consisted of screening approximately 500 individuals of Ashkenazi descent with 500 control individuals. Approximately 17,000 candidate single nucleotide polymorphisms (SNPs) suspected of yielding susceptibility to colorectal cancer were identified. The identified SNPs were shown to match allele frequency within the pools of individuals. 5000 of the top SNP candidates were validated in a separate set of colorectal cancer cases (e.g., approximately 1500 individuals diagnosed with colorectal cancer) and controls (e.g., approximately 1500 individuals not diagnosed with colorectal cancer). Genetic variation in 8 markers, including 6 genes, were shown to significantly modify the risk of colorectal cancer. Genetic variation at each of these markers and their associated genes were shown to predict risk of colorectal cancer and be useful for colorectal cancer diagnoisis and prognosis. Replication and validation of results were available for a limited subset of SNPs derived from case-control studies in Israel, Spain, and Germany, in addition to the original analytic dataset of all 8 markers and 6 genes. The following single nucleotide polymorphisms were shown to be associated with risk of colorectal cancer: dbSNP rsID 544670, associated with a 41% increase in risk (OR=1.41, 95% confidence interval 1.22-1.61 p=0.000002) among 2,000 cases and 2,000 controls, rsID16931815, associated with a 13% decrease in risk (OR=0.87, 95% confidence interval 0.81-0.93, p=0.00012) among 6,994 cases and 7,178 controls dbSNP rsID 10210149, associated with a 11% decrease in risk (OR=0.89, 95% confidence interval 0.84-0.93, p=0.0000015) among 7,258 cases and 7,201 controls, dbSNP rsID 17029, associated with a 25% increase in risk (OR=1.25, 95% confidence interval 1.12-1.39, p=0.000056) among 2,000 cases and 2,000 controls, dbSNP rsID10194088, associated with a 19% increase in risk (OR=1.19, 95% confidence interval 1.09-1.30, p=0.00012) among 2,000 cases and 2,000 controls, dbSNP rsID 2977269, associated with a 22% increase in risk (OR=1.22, 95% confidence interval 1.09-1.39, p=0.00072) among 2,000 cases and 2,000 controls, dbSNP rsID 883992, associated with a 20% increase in risk (OR=1.20, 95% confidence interval 1.09-1.33, p=0.0004) among 2,000 cases and 2,000 controls, dbSNP rsID 7896882, associated with a 35% increase in risk (OR=1.35, 95% confidence interval 1.16-1.57, p=0.000099) among 2,000 cases and 2,000 controls. Genetic variation in and around genes STK38L (p=0.00012), GPR45 (p=0.0000015), TGFBRAP1 (p=0.0000015), PADI3 (p=0.0018), IBRDC1 (p=0.0028), and GRK5 (p=0.0042) were shown to be significantly predictive of risk of colorectal cancer. These findings were validated in other large samples of individuals.

We claim:

1. A method of characterizing colorectal cancer in a subject, comprising: providing a sample from a subject; and determining genetic variation associated with one or more of said subject's STK38L, GPR45, TGFBRAP1, PADI3, IBRDC1, and GRK5 genes to characterize colorectal cancer.

2. The method of claim 1, wherein said genetic variation is associated with one or more of a single nucleotide polymorphism selected from the group consisting of rsID 544670, rsID16931815, rsID 10210149, rsID 17029, rsID 10194088, rsID 2977269, rsID 883992, and rsID 7896882.

3. The method of claim 2, wherein a genetic variation associated with the presence of one or more single nucleotide polymorphisms selected from the group consisting of rsID 544670, rsID 17029, rsID 10194088, rsID 2977269, rsID 883992, and rsID 7896882 indicates an increased susceptibility to colorectal cancer.

4. The method of claim 2, wherein a genetic variation associated with the presence of one or more single nucleotide polymorphisms selected from the group consisting of rsID 16931815, and rsID 10210149 indicates a decreased susceptibility to colorectal cancer.

5. The method of claim 1, wherein said determining genetic variation within one or more of said subject's STK38L, GPR45, TGFBRAP1, PADI3, IBRDC1, and GRK5 genes comprises the use of a nucleic acid based detection assay.

6. The method of claim 1, wherein said subject is not previously diagnosed with colorectal cancer.

7. The method of claim 1, wherein said characterizing colorectal cancer comprises determining the risk of developing colorectal cancer.
